(19) United States
(12) Patent Application Publication
(10) Pub. No.: US 2011/0256305 A1
(43) Pub. Date: Oct. 20, 2011

(54) Turbine Coating Apparatus And Spray Gun Assembly Therefor

(75) Inventors: Wilfrid Jacques, Longueuil (CA); Francine Mondou, Blainville (CA)

(73) Assignees: HARMONIUM INTERNATIONAL INC., Mirabel, QC (CA); Wilfrid Jacques, Longueuil, QC (CA)

(21) Appl. No.: 13/141,309

(22) PCT Filed: Dec. 23, 2008
(86) PCT No.: PCT/CA08/02292

§ 371 Date: Jun. 21, 2011

(51) Int. Cl. A61K 9/16; A23G 3/26; B05D 7/00; B05C 5/00

(52) U.S. Cl. 427/2.18; 118/303; 118/19; 427/212

(57) ABSTRACT

The present invention generally relates to a turbine coating apparatus and a spray gun assembly therefor. More specifically, the present invention relates to a spray gun assembly comprising a gun support mountable to the turbine coating apparatus and at least one spray gun mounted to the gun support. The spray gun is adapted for providing a spray of a coating substance onto a cluster of solid forms to be coated in the apparatus, where the spray defines a spray angle of less than 90 degrees relative to the coating plane defined by the cluster of solid forms in movement.

### FIELD OF THE INVENTION

The present invention generally relates to a turbine coating apparatus and to a spray gun assembly therefor. More specifically, the present invention relates to a spray gun assembly comprising a gun support mountable to the turbine coating apparatus and at least one spray gun mounted to the gun support. The spray gun is adapted for providing a spray of a coating substance onto a cluster of solid forms to be coated in the apparatus, where the spray defines a spray angle of less than 90 degrees relative to the coating plane defined by the cluster of solid forms in movement.

### BACKGROUND OF THE INVENTION

Probiotics are dietary supplements containing potentially beneficial yeasts or bacteria such as Lactic acid bacteria (LAB). When administered in sufficient amounts, probiotic micro-organisms confer health benefits on the host, including managing lactose intolerance, preventing colon cancer, lowering cholesterol and preventing gastrointestinal infections.

As most of the beneficial actions of probiotics take place in the gut, bacteria's survival strengths and reproducibility capacities are the key criteria when selecting bacterial strains for the production of probiotics. The survival of micro-organisms is highly dependent on gastric resistance during passage through the stomach's acidic environment toward the gut. Therefore, resistance to acidic pH is one of the main factors to consider when selecting the most potent probiotic bacteria.

While administration of probiotics can be made through consumption of probiotic-containing food such as yogurt, alternative administration modes that contribute to increase the survival rate of the probiotic bacteria during the passage through the stomach have been envisioned. One of such alternative administration mode known in the art resides in providing a probiotic culture packaged in a capsule, which capsule is further coated to resist the harsh acidic environment of the stomach.

Capsules, tablets and other solid forms generally disintegrate in the stomach and, less frequently, disintegration will be completed in the upper part of the small intestine. For these solid forms to resist to the acidic environment of the stomach and be disintegrated in the small intestine, where conditions are alkaline, they must be coated with an enteric coating.

Enteric coating may be provided on solid forms using coating apparatuses. Prior art teaches multiple coating apparatus configurations, including top-spray and bottom-spray fluid bed coaters, Wurster coater, fluid bed coating apparatus and conventional, imperforated pan coating apparatus and perforated turbine coating apparatus, the later being generally preferred for coating capsules for efficiency purposes.

A typically turbine coating apparatus includes a perforated drum mounted for rotation about a horizontal rotation axis in a housing. A drive assembly is also provided for driving rotation of the perforated drum. The turbine coater further includes an air intake mounted in the periphery of the perforated drum for introducing hot air into the drum, through the perforations thereof, and an air exhaust for collecting air, particles, dust and volatilized solvents from the drum. The air exhaust is mounted in the periphery of the perforated drum, generally in a position radially opposed to the air intake.

A spray gun assembly is provided for feeding the coating material in the perforated drum and to uniformly coat the capsules, granules, tablets and other solid forms. A typical spray gun assembly includes a main arm mounted to the housing of the apparatus by one end, a gun mounting bracket mounted to the other end of the main arm and a plurality of spray guns mounted to the bracket. The spray gun assembly is adapted for positioning the bracket and the spray guns inside the drum in operation and to remove the same from the drum upon completion of the coating process. As such, the main arm may include a swing arm.

Baffles and longitudinal anti-slides (or tumbling bars) are preferably provided on the interior face of the perforated drum for controlling the movement of tablets, capsules and other solid forms being coated while the perforated drum of the turbine coating apparatus is rotated. More specifically, the baffles and the anti-slides contribute to gather the solid forms to be coated in the bottom left portion of the perforated drum in rotation while such drum is rotated clockwise, thereby forming a cluster or bed of solid forms. The spray guns are typically configured to spray the coating substance towards the upper portion of the bed or cluster of solid forms while the capsules, tablets and the like are falling down towards the bottom of the drum. This upper portion, often referred to as the upper third of the cluster, tends to define an angular plane during operation of the turbine coating apparatus, which plane will serve as reference for positioning the spray gun assembly. In most cases, the spray guns are configured to spray the coating substance perpendicularly to the angular plane of the upper third of the cluster of solid forms, i.e. at an angle of about 90 degrees relative to said plane (as best shown in FIGS. 8A, 8B and 8C).

The coating process using such a turbine coating apparatus involves multiple interdependent parameters which may affect the amount of coating substance required, the time required for the coating process and the overall efficiency of the process. More specifically, coating applied on solid forms is usually defined by the amount of coating solution used expressed as a percentage of the total weight of solid forms to be coated. This weight gain value is a theoretical desired value as preset by a user of the turbine coating apparatus. For instance, the turbine coating apparatus may be preset to obtain capsules having a coating layer providing a weight gain of about 6%.

To further appreciate or validate the characteristics of coated solid forms, samples of coated solid forms may be weighted in order to establish an empirical weight gain value. This empirical weight gain value may further be compared with the theoretical weight gain value to determine the efficiency of the coating process and the quantity of coating material lost in the coating process.

The efficiency of the coating process also depends upon the distribution of the coating substance over the solid forms to be coated. For a same theoretical weight gain, the coating substance distribution may vary substantially over the solid forms. This parameter is particularly important with hard shell capsules, also referred to as two-piece capsules. A hard shell capsule is a pharmaceutical element made from two hollow parts, namely a body and a cap, filled with drugs or the like and joined to one another by a circumferential joint. Therefore, with hard shell capsules, the enteric coating also plays a role in sealing the circumferential joint between the body and the cap. It is noteworthy that for a same theoretical weight gain, the quality of sealing of capsules and the variation of the quality of sealing may vary greatly. The quality of sealing may be defined as the ratio of the total sealed portion of the circumferential joint of the hard shell capsule relative to the total circumference of the joint of the hard shell capsule following a coating operation. A high value of quality of sealing of hard shell capsules is desirable to prevent the probiotic culture from being released prior to reaching the gut during consumption.

The variability of quality of sealing may be established from values of quality of sealing observed in samples of capsules, usually by calculating a relative standard deviation value. A high value of relative standard deviation implies that the quality of sealing of capsules from a same lot varies greatly from one capsule to another. Inversely, a low value of relative standard deviation implies that the quality of sealing of capsules from a same lot does not vary much from one capsule to another. A low value is therefore desirable to achieve consistent coating results using a turbine coating apparatus.

Therefore, it is desirable to obtain better quality of sealing and low standard deviation values with a minimal weight gain. However, the turbine coating apparatus configurations and of the spray guns of the prior art tend not to be satisfactorily for coating and sealing capsules, especially when enteric coating is used. Indeed, the various interdependent parameters of the coating process lead either to the use of larger amounts of coating substance for obtaining a better quality of sealing or, alternatively, to a higher percentage of rejection of inadequately sealed capsules where lower amounts of coating substances are used.

To alleviate such drawbacks, coating methods of the prior art include sealing the joint between the body and the cap of the hard shell capsule prior to subject the capsules to the coating. Such sealing process is aimed at ensuring the hard shell capsule joint is appropriately sealed to prevent unwanted infiltration of gastric fluid within the capsule and premature degradation of the probiotics. The joint may be sealed by providing a sealing band or by a micro-spray sealing apparatus. Both the application of the sealing strips and the micro-spray sealing of the hard shell capsules requires specialized equipment and adds an additional step to the coating process, which therefore tends to slow down the overall coating process and tend to increase production costs.

Therefore, it would be desirable to be provided with a turbine coating apparatus and/or a spray gun assembly, which would contribute to reduce at least one of the above-mentioned drawbacks.

### SUMMARY OF THE INVENTION

According to one embodiment, there is provided a spray gun assembly for a turbine coating apparatus used for coating solid forms such as capsules, pellets and the like. The turbine coating apparatus is provided with a perforated drum rotatably mounted in a housing, the rotation of the perforated drum gathering solid forms in a cluster defining a coating plane.

According to this embodiment, the spray gun assembly comprises a gun support mountable to the turbine coating apparatus and at least one spray gun mounted to the gun support for providing a spray of a coating substance onto the cluster of solid forms. The at least one spray gun is further positionable into the perforated drum in rotation for the spray of a coating substance to define a spray angle of less than 90 degrees relative to the coating plane defined by the cluster of solid forms.

In accordance with another embodiment, there is provided a turbine coating apparatus for coating solid forms such as capsules, pellets and the like.

The turbine coating apparatus is provided with a perforated drum rotatably mounted in a housing, the rotation of the perforated drum gathering solid forms in a cluster defining a coating plane. The turbine coating apparatus further comprises a gun support mounted to the turbine coating apparatus and at least one spray gun mounted to the gun support for providing a spray of a coating substance onto the cluster of solid forms. The at least one spray gun is positioned into the perforated drum in rotation for the spray of a coating substance to define a spray angle of less than 90 degrees relative to the coating plane defined by the cluster of solid forms.

According to one aspect, the spray angle ranges from about 10 degrees to about 80 degrees.

According to another aspect, the spray angle ranges from about 15 degrees to about 50 degrees.

According to yet another aspect, the gun support comprises a first end mountable to the housing of the apparatus and a second end, the at least one spray gun being mounted between the first end and the second end of the gun support.

According to yet another aspect, the at least one spray gun is adjustably positionable between the first end and the second end of the gun support.

According to yet another aspect, the solid forms are selected from the group consisting of capsules, granules and tablets.

According to a further aspect, the capsules comprise hard shell capsules.

According to yet a further aspect, the hard shell capsules comprise vegetal capsules.

According to yet a further aspect, the hard shell capsules comprise gelatin capsules.

According to another aspect, the capsules comprise capsules housing a substance selected from the group consisting of a probiotic culture, a pharmaceutical compound, a nutraceutical, a dietary supplement, a vitamin and a veterinary compound.

According to yet another aspect, the coating substance is selected from the group consisting of a sub-coating substance, an enteric coating substance and a film coating.

According to yet another aspect, the at least one spray gun is selected from the group consisting of a Schlick #930/7-1 S35™ spray gun, a Schlick #970/7-1 S75™ spray gun and a Spraying System Co. #1/4 JAU-SS™ spray gun.

There is further provided a method for coating and sealing solid forms. In accordance with one embodiment, the method comprises providing a turbine coating apparatus comprising a perforated drum rotatably mounted in a housing and providing at least one spray gun positionable into the perforated drum for providing a spray of a coating substance. The method further comprises loading the solid forms into the perforated drum, urging rotation of the perforated drum, where the rotation of the perforated drum gathers solid forms in a cluster defining a coating plane. The method further comprises positioning the at least one spray gun into the perforated drum for the spray of a coating substance to define a spray angle of less than 90 degrees relative to the coating plane, providing the spray of a coating substance onto the cluster of solid forms until a predetermined amount of weight gain has been provided to the solid forms and collecting the coated solid forms.

According to one aspect, the method further comprises providing a gun support mounted to the turbine coating apparatus, the at least one spray gun being mounted to the gun support.

According to another aspect, the gun support comprises a first end mountable to said housing of the apparatus and a second end, the at least one spray gun being mounted between the first end and the second end of the gun support.

According to yet another aspect, positioning the at least one spray gun comprises adjustably positioning the at least one spray gun between the first end and the second end of the gun support.

According to yet another aspect, positioning the at least one spray gun further comprises positioning the at least one spray gun eccentrically relative to the rotation axis of the perforated drum.

According to yet another aspect, loading the solid forms comprises removing the gun support from the turbine coating apparatus for facilitating access to the perforated drum and mounting the gun support to the turbine coating apparatus once the solid forms have been loaded into the perforated drum.

According to yet another aspect, the coated solid forms collecting comprises removing the gun support from the turbine coating apparatus for facilitating access to the perforated drum and removing the coated solid forms from the perforated drum.

According to one aspect, the spray angle comprises a spray angle ranging from about 10 degrees to about 80 degrees.

According to another aspect, the spray angle comprises a spray angle ranging from about 15 degrees to about 50 degrees.

According to yet another aspect, the solid forms are selected from the group consisting of capsules, granules and tablets.

According to a further aspect, the capsules comprise hard shell capsules.

According to yet a further aspect, the hard shell capsules comprise vegetal capsules.

According to yet a further aspect, the hard shell capsules comprise gelatin capsules.

According to another aspect, the capsules comprise capsules housing a substance selected from the group consisting of a probiotic culture, a pharmaceutical compound, a nutraceutical, a dietary supplement and a vitamin.

According to yet another aspect, the coating substance is selected from the group consisting of a sub-coating substance, an enteric coating substance and a film coating.

According to yet another aspect, the at least one spray gun is selected from the group consisting of a Schlick #930/7-1 S35™ spray gun, a Schlick #970/7-1 S75™ spray gun and a Spraying System Co. #1/4 JAU-SS™ spray gun.

These and other objects, advantages and features of the present invention will become more apparent to those skilled in the art upon reading the details of the invention more fully set forth below.

### BRIEF DESCRIPTION OF THE DRAWINGS

Having thus generally described the nature of the invention, reference will now be made to the accompanying drawings, showing by way of illustration an illustrative embodiment thereof, and in which:

FIG. 1 is a front left perspective view of a turbine coating apparatus in accordance with one embodiment of the present invention;

FIG. 2 is a front elevation view of the turbine coating apparatus shown in FIG. 1 with a spray gun assembly in accordance with one embodiment of the present invention;

FIG. 3 is a left side view of the turbine coating apparatus shown in FIG. 2, with the left side wall and the perforated drum partially cross-sectioned for showing the interior of the perforated drum;

FIG. 4 is an enlarged front left perspective view of the turbine coating apparatus shown in FIG. 2;

FIG. 5 is another enlarged front left perspective view of the turbine coating apparatus shown in FIG. 2, with the spray gun assembly removed for better showing the baffles and the anti-slides;

FIG. 6 is an exploded view of a spray gun assembly for the turbine coating apparatus in accordance with one embodiment of the present invention;

FIG. 7 is a further enlarged front left perspective view of the turbine coating apparatus shown in FIG. 2, with a cluster of solid forms therein;

FIG. 8A is a cross-section of a turbine coating apparatus in accordance with the prior art, showing the perforated drum in rotation and the position of the spray gun assembly with respect to the plane defined by the cluster of solid forms contained therein;

FIG. 8B is another cross-section view of the turbine coating apparatus shown in FIG. 8A with the cluster of solid forms removed for better showing the angle between the spray of the spray gun assembly and the plane defined by the cluster of solid forms;

FIG. 8C is an enlarged view of the turbine coating apparatus shown in FIG. 8A for better showing the angle between the spray of the spray gun assembly and the plane defined by the cluster of solid forms;

FIG. 9A is an enlarged, cross-section of the turbine coating apparatus, shown in FIG. 3, taken along cross-section line IX-IX, showing the perforated drum in rotation and the position of the spray gun assembly with respect to the plane defined by the cluster of solid forms contained therein;

FIG. 9B is another cross-section view of the turbine coating apparatus, shown in FIG. 9A with the cluster of solid forms removed for better showing the angle between the spray of the spray gun assembly and the plane defined by the cluster of solid forms;

FIG. 9C is an enlarged view of the turbine coating apparatus shown in FIG. 9A for better showing the angle between the spray of the spray gun assembly and the plane defined by the cluster of solid forms;

FIG. 10A is a comparative graph showing the actual weight gain of capsules in relation to the theoretical weight gain thereof, where capsules were subject to coating according a method of the prior art (Lot 1A) and subject to coating according to one embodiment of the present invention (Lot 1B);

FIG. 10B is a comparative graph showing the quality of sealing of capsules in relation to theoretical weight gain thereof, where capsules were subject to coating according a method of the prior art (Lot 1A) and subject to coating according to one embodiment of the present invention (Lot 1B);

FIG. 10C is a bar chart representing the variability of the quality of sealing of capsules in relation to theoretical weight gain thereof, where capsules were subject to coating according a method of the prior art (Lot 1A) and subject to coating according to one embodiment of the present invention (Lot 1B);

FIG. 11A is a comparative graph showing the actual weight gain of capsules in relation to the theoretical weight gain thereof, where capsules were subject to coating according a method of the prior art (Lot 2A) and subject to coating according to one embodiment of the present invention (Lot 2B);

FIG. 11B is a comparative graph showing the quality of sealing of capsules in relation to theoretical weight gain thereof, where capsules were subject to coating according a method of the prior art (Lot 2A) and subject to coating according to one embodiment of the present invention (Lot 2B);

FIG. 11C is a bar chart representing the variability of the quality of sealing of capsules in relation to theoretical weight gain thereof, where capsules were subject to coating according a method of the prior art (Lot 2A) and subject to coating according to one embodiment of the present invention (Lot 2B);

FIG. 12A is a comparative graph showing the actual weight gain of capsules in relation to the theoretical weight gain thereof, where capsules were subject to coating according a method of the prior art (Lot 3A) and subject to coating according to one embodiment of the present invention (Lot 3B);

FIG. 12B is a comparative graph showing the quality of sealing of capsules in relation to theoretical weight gain thereof, where capsules were subject to coating according a method of the prior art (Lot 3A) and subject to coating according to one embodiment of the present invention (Lot 3B); and

FIG. 12C is a bar chart representing the variability of the quality of sealing of capsules in relation in relation to theoretical weight gain thereof, where capsules were subject to coating according a method of the prior art (Lot 3A) and subject to coating according to one embodiment of the present invention (Lot 3B).

### DESCRIPTION OF THE ILLUSTRATIVE EMBODIMENTS

The description which follows, and the embodiments described therein are provided by way of illustration of an example, or examples of particular embodiments of principles and aspects of the present invention. These examples are provided for the purpose of explanation and not of limitation. In the description that follows, like parts are marked throughout the specification and the drawings with the same respective reference numerals.

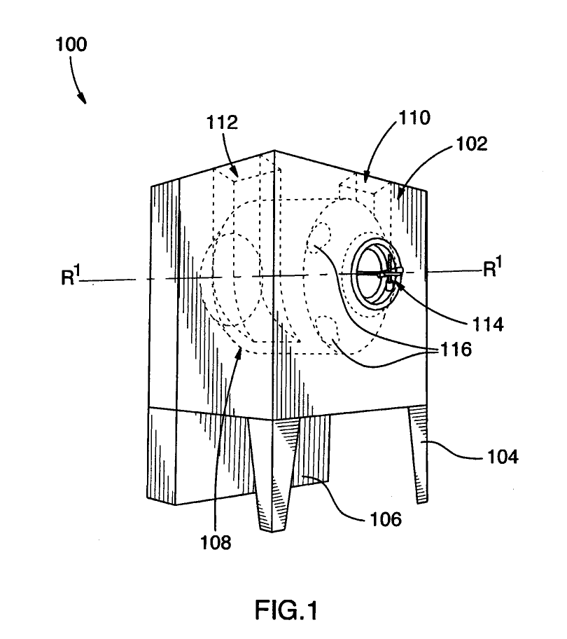

With reference to FIG. 1, turbine coating apparatus will be described in accordance with one embodiment of the present invention, using the reference numeral 100. The turbine coating apparatus 100 is adapted for coating solid forms such as capsules, tablets, pellets and the like. A person skilled in the art will appreciate that the term “capsule” as intended herein may include soft capsules and hard shell capsules. In one embodiment of the present invention, the turbine coating apparatus 100 is used for coating hard shell (or two-piece) capsules containing probiotic cultures with an enteric coating. The term “enteric coating” is generally intended to mean any barrier applied to a composition administered orally, which barrier or coating controls the release location of such composition in the digestive tract. More specifically, enteric coating relates to any coating that will prevent the release of the composition before it reaches the small intestine of the individual to which such composition is administered. A person skilled in the art will nevertheless understand that the turbine coating apparatus 100 may find use in a number of applications, including coating of pharmaceutical compounds, nutraceuticals, dietary supplements, vitamins, veterinary compounds, fertilizer compositions and the like. A person skilled in the art will further appreciate that the use of the turbine coating apparatus 100 is not limited to coating of hard shell capsules but rather extends to any type of solid forms or particle material that may require coating, as it will become apparent below.

The turbine coating apparatus 100 comprises a housing 102 mounted on legs 104 and 106. Rotatably mounted in the housing 102 is a perforated drum 108 configured for rotating about a generally horizontal rotation axis R¹-R¹, the rotation of the perforated drum 108 being driven by a drive assembly (not shown). In one embodiment, the drive assembly comprises an electric motor (not shown) operatively coupled to the perforated drum 108 by a transmission (not shown). Preferably, the drive assembly rotates the perforated drum 108 in a clockwise direction. Alternatively, the drive assembly may rotate the perforated drum 108 in a counter-clockwise direction, or selectively in both clockwise and counter-clockwise directions.

The turbine coating apparatus 100 further comprises an air intake assembly 110 mounted in the housing 102, in the periphery of the perforated drum 108, for introducing a flow of warm air into the perforated drum 108 and an air exhaust assembly 112. The air exhaust assembly 112 is also mounted in the housing 102, in the periphery of the perforated drum 108, for collecting air from the perforated drum 108 and particles, dust and volatilized solvents contained therein, as it will become apparent below.

The turbine coating apparatus 100 also comprises a spray gun assembly 114 for feeding the coating material in the perforated drum 108 and to uniformly coat the hard shell capsules during operation of the turbine coating apparatus 100. A deflector assembly 116 associated within the perforated drum 108 and configured for directing the movement of the capsules toward the spray gun assembly 114 is also provided as it will become apparent below.

In one embodiment of the present invention, the turbine coating apparatus 100 may correspond to a turbine coating apparatus such as those known in the art, with the exception that the spray gun assembly 114 and the deflector assembly 116 have been modified to improve the sealing and coating capabilities of such turbine coating apparatus. For instance, the housing 102, the perforated drum 108, the air intake assembly 110 and the air exhaust assembly 112 may correspond to those of the Labcoat II™ commercialized by O'Hara Technologies (Richmond Hill, ON, Canada). As it will become apparent below, a person skilled in the art will appreciate that the spray gun assembly 114 and the deflector assembly 116 of the present invention could be provided on any other type of turbine coater.

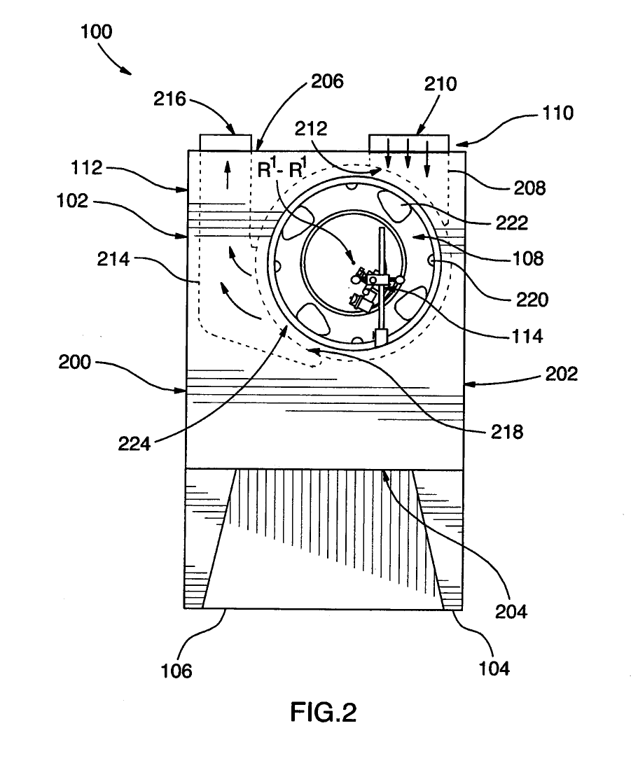

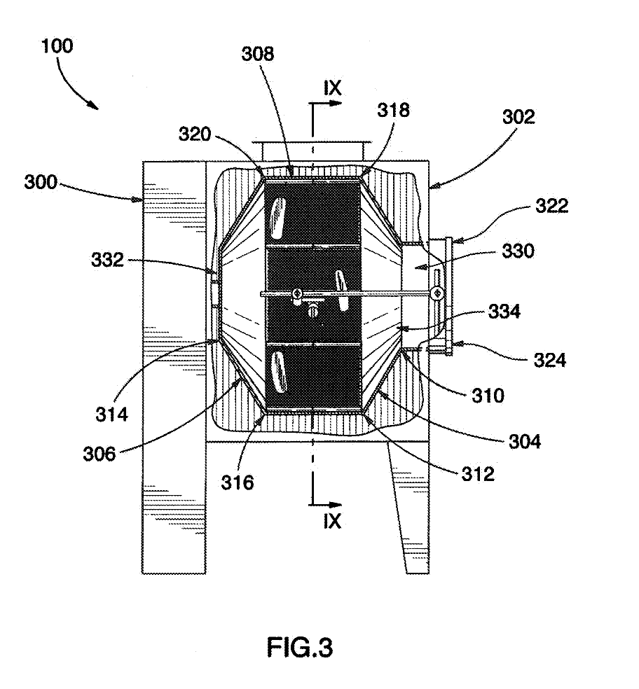

Now referring to FIGS. 2 and 3, the housing 102 comprises a back wall 300, a front wall 302 and a pair of spaced-apart side walls 200, 202. The housing further comprises a bottom wall 204 to which are connected the legs 104, 106, and a top wall 206. The legs 104, 106 are provided for positioning the housing 102 above the surface of the ground, at an elevation that is ergonomically satisfactory for accessing the interior of the perforated drum 108 for filling the same with hard shell capsules to be coated and collect the coated hard shell capsules upon completion of the coating process. A person skilled in the art will thus appreciate that the housing 102 and the legs 104, 106 may be configured differently without departing from the scope of the invention.

Provided on the front wall 302 of the housing 102 is a circular opening 330 for accessing the perforated drum 108, as it will become apparent below. The perforated drum 108 comprises a front frustro-conical wall 304, a back frustro-conical wall 306 spaced-apart front the front frustro-conical wall 304 and a cylindrical wall 308 extending between the back and front walls 306, 304. The front frustro-conical wall 304 comprises a smaller diameter front edge 310 and a larger diameter back edge 312. Similarly, the back frustro-conical wall 306 comprises a smaller diameter back edge 314 and a larger diameter front edge 316. The perforated drum 108 is provided with a back circular wall 332 adjacent to the smaller diameter back edge 314 of the back frustro-conical wall 306 for closing the back end of the perforated drum 108 while the smaller diameter front edge 310 of the front frustro-conical wall 304 defines an opening 334 for accessing the interior of the perforated drum 108 for feeding the capsules to be coated or collecting the capsules upon completion of the coating process. In one embodiment, the opening 334 of the perforated drum 108 has a diameter corresponding generally to the diameter of the opening 330 defined on the front wall 302 of the housing 102.

The cylindrical wall 308 comprises a circular front edge 318 connected to the larger diameter back edge 312 of the front frustro-conical wall 304 and a circular back edge 320 connected to the larger diameter front edge 316 of the back frustro-conical wall 306. As it will be appreciated by a person skilled in the art, the configuration of the perforated drum 108, and more specifically of the front and back frustro-conical walls 304, 306, contributes to direct and maintain the bed of capsules on the surface of the cylindrical wall 308 during the operation of the turbine coating apparatus 100. Further, the configuration of the front frustro-conical wall 306 contributes to prevent unwanted escape of the capsules through the opening 334 during the operation of the turbine coating apparatus 100.

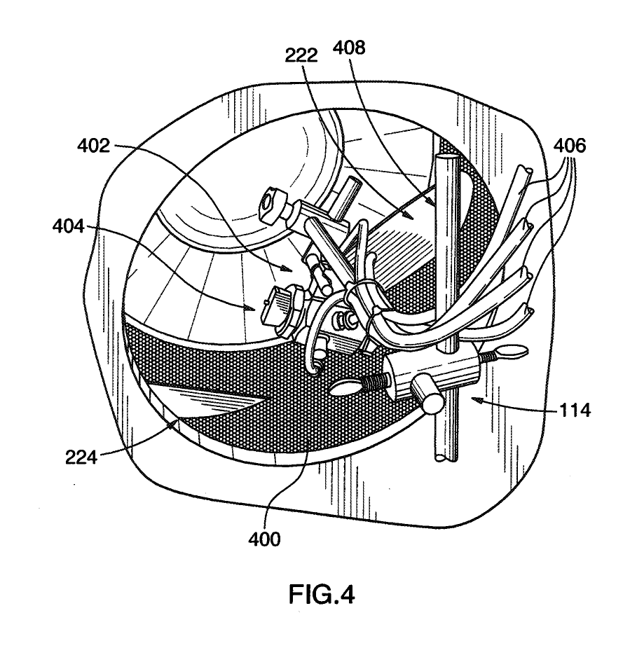

Provided on the cylindrical wall 308 of the perforated drum 108 is a plurality of perforations 400 (best shown in FIG. 4). In one embodiment, the perforations 400 are distributed between the front and back circular edges 318, 320 of the cylindrical wall 308 and are adapted for allowing the entry of the air from an air intake duct 208 and the exit of the exhaust air toward an air exhaust duct 214 while avoiding the passage therethrough of the hard shell capsules to be coated, as it will become apparent below. In this embodiment, each perforation 400 is a circular perforation having a diameter ranging from about 2 mm to about 4 mm.

The perforated drum 108 is rotatably mounted in the housing 102 and extends generally between the back wall 300 and the front wall 302 thereof. More specifically, the circular back wall 332 of the perforated drum 108 is positioned proximal to the back wall 300 of the housing 102 while the smaller diameter front edge 310 of the front frusto-conical wall 304 and the opening 334 defined thereby are located adjacent to the front wall 302 of the housing 102. In one embodiment, the opening 334 of the perforated drum 108 is aligned with the opening 330 of the housing for allowing convenient access to the interior of the perforated drum 108.

In one embodiment of the present invention, the circular back wall 332 of the perforated drum 108 is operatively connected to the back wall 300 of the housing 102 by a mounting assembly (not shown). Such a mounting assembly is known in the art and typically comprises a circular mounting bracket (not shown) matingly mounted to the circular back wall 332 of perforated drum 108. The circular mounting bracket (not shown) may be fastened to the circular back wall 332 of the perforated drum 108 using fasteners such as screws or, alternatively, be welded to the circular back wall 332 of the perforated drum 108 or fixed by any other suitable means.

In this embodiment, the back wall 300 of the housing 102 is provided with a generally circular opening (not shown) housing a bearing assembly (not shown), the opening and bearing assembly being in alignment with the axis of rotation R¹-R¹ of the perforated drum 108.

The mounting assembly (not shown) further comprises a drive shaft (not shown) extending between the circular mounting bracket (not shown) and the bearing assembly (not shown) of the housing 102. At one end thereof, the drive shaft (not shown) is provided with a drive gear (not shown) operatively coupled to a motor (not shown) by a chain (not shown). As such, actuation of the electric motor (not shown) provides rotation of the perforated drum 108. It will be appreciated by a person skilled in the art that the drive shaft (not shown) is positioned parallel to the axis of rotation R¹-R¹ of the perforated drum 108 such that when the electric motor (not shown) is actuated, the perforated drum 108 rotates with respect to the axis of rotation R¹-R¹.

The mounting assembly (not shown) is further provided with a bearing assembly (not shown) known in the art concentrically mounted on the drive shaft (not shown) and circumferentially mounted inside the opening located on the back wall 300 of the housing 102. A person skilled in the art will appreciate that the bearing assembly (not shown) reduces friction of the drive shaft (not shown) inside the opening located on the back wall 300 of the housing 102 during rotation of the mounting assembly (not shown).

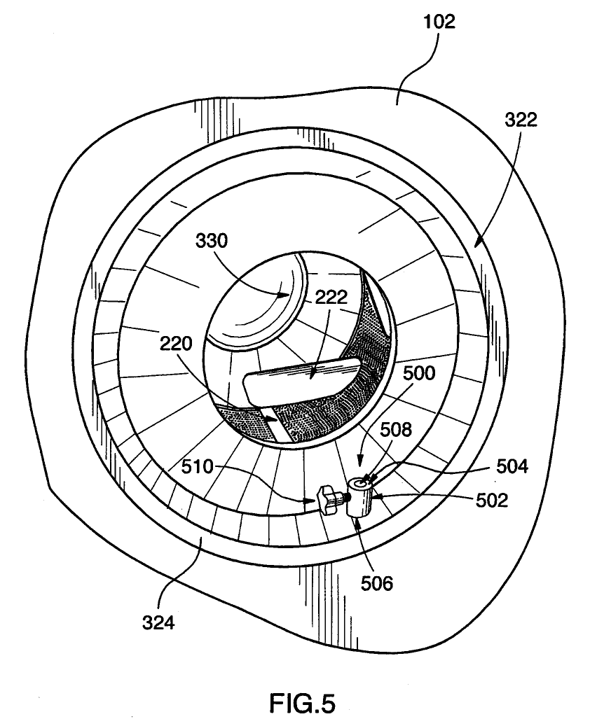

The housing 102 is further provided with an annular protrusion 322 (best shown in FIGS. 3 and 5) extending from the front wall 302. The annular protrusion 322, having a diameter larger than the circular opening 330 of the housing 102, is positioned concentrically there around and comprises an inner curved surface 324 (best shown in FIGS. 3 and 5).

In one embodiment, the annular protrusion 322 and the front wall 302 of the housing 102 define an integral structure. In an alternative embodiment, the annular protrusion 322 may be securely fastened to the front wall 302 of the housing 102 using fasteners known to the skilled addressee such as screws, rivets or the like. In yet another embodiment, the annular protrusion 322 may be welded to the front wall 302 of the housing 102 using welding techniques known to the skilled addressee.

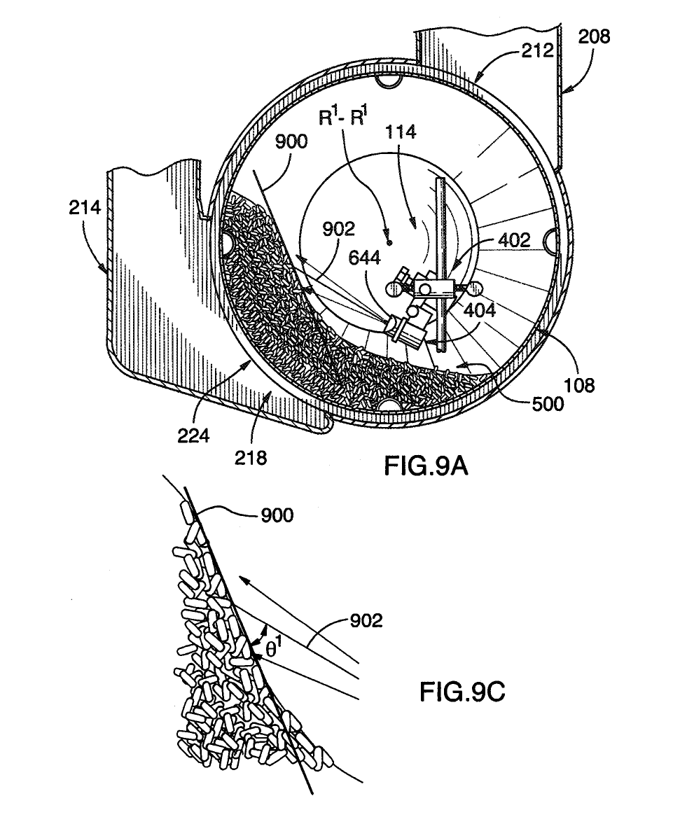

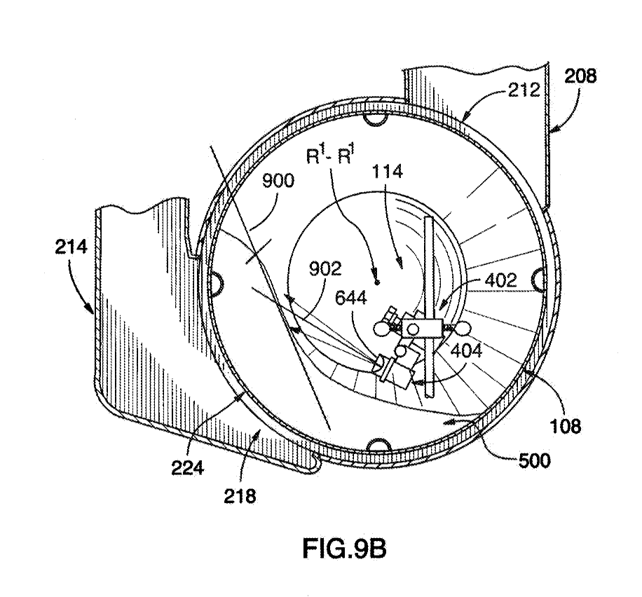

Now referring to FIGS. 2, 5 and 9A, a plurality of longitudinal anti-slides 220 (also referred to as tumbling bars) and baffles 222 are mounted to the interior face of the perforated drum 108. The anti-slides and the baffles 220, 222 contribute to gather the capsules to be coated in the bottom left portion 224 of the perforated drum 108 in rotation while such perforated drum 108 is rotated clockwise (as best shown in FIG. 9A). Further, the baffles 222 and anti-slides 220 contribute to the tumbling of the hard shell capsules during the rotation of the drum, thus improving the overall sealing process, as it will become apparent below. The upper portion of the cluster of hard shell capsules (i.e. the upper third) defines an angular plane 900 during operation of the turbine coating apparatus 100, which plane will further be used as reference for positioning the spray gun assembly 114, as it will become apparent below.

When the perforated drum 108 is rotated counter-clockwise, the baffles and the anti-slides 220, 222 are mounted to the interior face of the perforated drum 108 such that the baffles and the anti-slides 220, 222 contribute to gather capsules to be coated in the bottom right portion of the perforated drum 108 in rotation.

Defined in the top wall 206 of the housing 102 is an air intake opening (not shown) adapted for receiving therethrough the air intake duct 208. The air intake duct 208 comprises a first open end 210 extending through the air intake opening, towards the exterior of the housing 102, and a second open end 212, located inside the housing 102, adjacent to the perforated drum 108. The second end 212 of the air intake duct 208 is sized and shaped to extend proximal to the perforated drum 108 and to match the curve of the cylindrical wall 308 for efficiently introducing a flow of air in the perforated drum 108 through the perforations 400. As it will be appreciated by a person skilled in the art, while being proximal to the cylindrical wall 308 of the perforated drum 108, the second end 212 of the air intake duct 208 is detached from the perforated drum 108 so as to allow its free rotation during the operation of the turbine coating apparatus 100.

For introducing air into the perforated drum 108, the air intake duct 208 is coupled to a pump (not shown). The air intake duct 208 is further coupled to heating elements (not shown) for warming the air introduced in the perforated drum 108 to a predetermined temperature to facilitate setting of the coating onto the capsules. In an alternative embodiment, the air intake duct 208 may further be coupled to a filtering system (not shown) for removing unwanted airborne particles in suspension from the air introduced in the perforated drum 108.

The top wall 206 of the housing 102 also comprises an air exhaust opening (not shown) adapted for allowing the passage therethrough of the air exhaust duct 214. Similarly to the air intake duct 208, the air exhaust duct 214 comprises a first open end 216 extending through the air exhaust opening, toward the exterior of the housing 102, and a second open end 218. The second end 218 of the air exhaust duct 214 is sized and shaped to extend proximal to the perforated drum 108 and to match the curve thereof for efficiently collecting air and contaminants (i.e. dust, volatilized solvent and the like) from the perforated drum 108. The second end 218 of the air exhaust duct 214 is detached from the perforated drum 108 so as to allow its free rotation during the operation of the turbine coating apparatus 100.

The second end 212 of the air intake duct 208 and the second end 218 of the air exhaust duct 214 are preferably located in radially opposed directions relative to the cylindrical wall 308 of the perforated drum 108 so as to maximise air circulation through the bed of capsules being coated. For instance, in one embodiment, the second end 212 of the air intake duct 208 adjoins the cylindrical wall 308 of the perforated drum 108 on the upper right portion thereof while the second end 218 of the air exhaust duct 214 adjoins the cylindrical wall 308 of the perforated drum on the bottom left portion 224 thereof (as seen on FIG. 2). In an alternative embodiment, the second end 212 of the air intake duct 208 adjoins the cylindrical wall 308 of the perforated drum 108 on the upper right portion thereof while the second end 218 of the air exhaust duct 214 adjoins the cylindrical wall 308 of the perforated drum 108 on the top left portion thereof

A person skilled in the art will appreciate that the second end 218 of the air exhaust duct 214 is located near the bed of capsules to be coated to efficiently collect air, particles, dust and volatilized solvents from the perforated drum 108 when the turbine coating apparatus 100 is in operation. In the present embodiment, as the capsules to be coated are gathered in the bottom left portion 224 of the perforated drum 108 due to the clockwise rotation of the perforated drum 108, the second end 218 of the air exhaust duct 214 adjoins the cylindrical wall 308 of the perforated drum 108 on the left side of the turbine coating apparatus 100 while the second end 212 of the air intake duct 208 adjoins the cylindrical wall 308 of the perforated drum 108 on the right side of the turbine coating apparatus 100.

In an embodiment where the perforated drum 108 is rotated counter-clockwise, the capsules to be coated are gathered in the bottom right portion of the perforated drum 108. In such an embodiment, the position of the air exhaust duct 214 and the air intake duct 208 are mirrored over the axis of rotation R¹-R¹, the second end 218 of the air exhaust duct 214 adjoining the cylindrical wall 308 of the perforated drum 108 on the right side of the turbine coating apparatus 100 and the second end 212 of the air intake duct 208 adjoining the cylindrical wall 308 of the perforated drum 108 on the left side of the turbine coating apparatus 100.

A person skilled in the art will appreciate that numerous configurations of air intake duct 208 and air exhaust duct 214 are possible and the spray gun assembly 114 of the present invention may be adapted to such different configurations in accordance with the direction of rotation of the perforated drum 108, as it will become apparent below.

Having described the general configuration of the perforated drum 108 and the housing 102, the spray gun assembly 114 will now be described with reference to FIGS. 4, 5, 6 and 7. The spray gun assembly 114 comprises a gun support 402 positionable inside the perforated drum 108 and a spray gun 404 mounted to the gun support 402 and adapted for spraying coating on the capsules during the operation of the turbine coating apparatus 100. The spray gun assembly 114 further comprises a plurality of tubes 406 extending between the spray gun 404 and coating supply and air supply sources (not shown) for conveying air and coating substance to the spray gun 404, as it will become apparent below.

In one embodiment, the gun support 402 is provided with a lower mounting element 500 securely mounted on the inner curved surface 324 of the annular protrusion 322 (best shown in FIG. 5). The lower mounting element 500 comprises a monolithic cylinder 502 having a first upper end 504 and an opposite, second lower end 506 mounted to the inner curved surface 324 of the annular protrusion 322. In a preferred embodiment, the second end 506 of the monolithic cylinder 502 may be welded, bolted or screwed onto the inner curved surface 324 of the annular protrusion 322 using techniques known to the skilled addressee.

The monolithic cylinder 502 is further provided with a central hole 508 axially extending between the first upper end 504 and the second lower end 506 of the monolithic cylinder 502. The central hole 508 is adapted for receiving therein the gun support 402. For securing the gun support 402 to the lower mounting element 500, a lock knob 510 is provided.

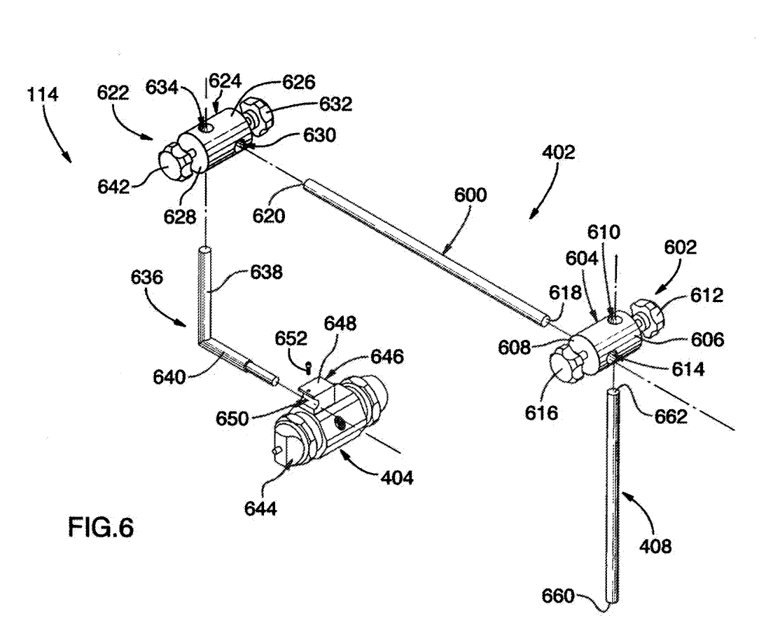

Now turning to FIG. 6, the gun support 402 comprises a vertical rod member 408 having a first lower end 660 adapted to engage the central hole 508 of the monolithic cylinder 502 and a second, opposed upper end 662. Once the gun support 402 is engaged in the central hole 508 of the monolithic cylinder 502 and positioned for operation of the turbine coating apparatus 100, the second end 662 of the vertical rod member 408 extends above the rotation axis R¹-R¹ of the perforated drum 108.

The gun support 402 further comprises a second, horizontal rod member 600 adjustably mounted to the first rod member 408 by a slidable connector 602, perpendicularly thereto. More specifically, the slidable connector 602 comprises a monolithic cylinder 604 made from a rigid material having a first end 606 and a second opposed end 608. Proximal to the first end 606 thereof, the monolithic cylinder 604 is provided with a first, vertically extending hole 610 configured for receiving therein the vertical rod member 408 and allowing vertical movement of the connector 602 relative to the vertical rod member 408. A lock screw 612 is provided at the first end 606 of the monolithic cylinder 604 for locking the connector 602 into position relative to the vertical rod member 408.

The monolithic cylinder 604 is further provided with a second, horizontally extending hole 614 configured for slidably receiving therein the second horizontal rod member 600 and allowing horizontal adjustment thereof. A second lock screw 616 is provided at the second end 608 of the monolithic cylinder 604 for locking the second, horizontal rod member 600 into position relative to the monolithic cylinder 604 and the vertical rod member 408.

The second rod member 600 comprises a first, outer end 618 received in the second hole 614 of the monolithic cylinder 604 and a second, inner end 620. During the operation of the turbine coating apparatus 100, the second inner end 620 of the second rod member 600 is located inside the perforated drum 108. In one embodiment, the gun support 402 is configured such that the second rod member 600 is positioned parallel to the rotation axis R¹-R¹ of the perforated drum 108, but offset thereof or, in other words, eccentrically relative to the rotation axis R¹-R¹ of the perforated drum 108. More specifically, where the turbine coating apparatus 100 is configured for the perforated drum 108 to rotate clockwise, the second rod member 600 is positioned slightly below and slightly on the right side of the rotation axis R¹-R¹ (as best shown in FIG. 9A). Where the perforated drum 108 is configured to rotate counter clockwise, the second rod member 600 may be positioned slightly below and slightly on the left side of the rotation axis R¹-R¹.

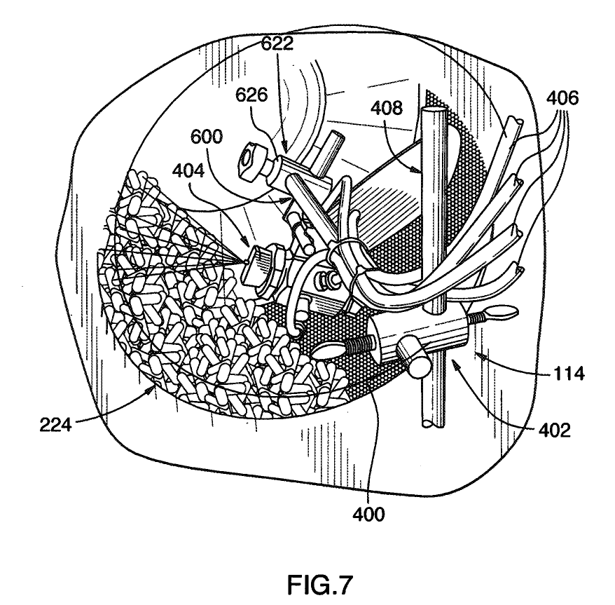

Provided at the second, inner end 620 of the second rod member 600 is a second slidable connector 622. The second slidable connector 622 is similar to the slidable connector 602 in that it comprises a monolithic cylinder 624 having a first end 626 and a second opposed end 628. Proximal to the first end 626, the second slidable connector 622 is provided with a first hole 630 extending horizontally and adapted for receiving therein the second rod member 600. A lock screw 632 is provided at the first end 626 of the second slidable connector 622 for locking the second slidable connector 622 into a desired position between the first end 618 and the second end 620 of the second rod member 600. As best shown in FIG. 7, the second slidable connector 622 is angled downwardly such that the first end 626 of the second slidable connector 622 is located slightly above the second end 628 of the second slidable connector 622. As the second rod member 600 is cylindrical and the first hole 630 of the second slidable connector 622 is also cylindrical, the angle of the second slidable connector 622 relative to the second rod member 600 can be adjusted.

The second slidable connector 622 further comprises a second hole 634, proximal to the second end 628. The second hole 634 extends perpendicular to the first hole 630 and is adapted for receiving therein an L-shape spray gun mounting member 636. The L-shaped mounting member 636 comprises a first portion 638 slidably receivable in the second hole 634 of the second slidable connector 622 and a second portion 640, perpendicular to the first portion 638 and configured to receive thereon the spray gun 404, as best described below. A second lock screw 642 is provided at the second end 628 of the monolithic cylinder 624 for locking the L-shaped mounting member 636 into position relative to the monolithic cylinder 624.

The spray gun 404 comprises a nozzle 644 provided with a mounting portion 646 for mounting the same to the second slidable connector 622. The nozzle 644 comprises a nozzle such as those known in the art, for instance a Schlick #930/7-1 S35™ nozzle, a Schlick #970/7-1 S75™ nozzle, a Spraying System Co. #1/4 JAU-SS™ nozzle or any similar nozzle. As such, the nozzle 644 does not require an exhaustive description. The mounting portion 646 of the spray gun 404 is mounted to the nozzle 644 and comprises an upwardly extending bracket 648 provided with a notch 650. The notch 650 is adapted for receiving therein the second portion 640 of the L-shaped mounting member 636. For maintaining the bracket 648 in position relative to the second portion 640 of the L-shaped mounting member 636, a locking screw 652 is provided.

A person skilled in the art will appreciate that the gun support 402 could be replaced by other devices. For instance, the gun support 402 could be replaced by a swing out arm similar to the one provided with the Fastcoat 60™ commercialized by O'Hara Technologies (Richmond Hill, ON, Canada). Such swing out arm comprises a support portion connected to the housing and an inner portion, pivotably mounted to the support portion. In such an embodiment, the inner portion would be provided with the spray gun 404 and would be movable inside and outside the perforated drum 108. Further, the number and the position of the spray gun 404 (relative to the front and back walls 304 and 306 of the perforated drum 108) could be different. For instance, instead of using a single spray gun, two spray guns could be mounted on the gun support 402 in a side-by-side relationship. Alternatively, instead of providing a gun support 402 as described above, one may find beneficial to use a manifold provided with a plurality of spray guns. It will be appreciated other configurations of the gun support 402 may be used to allow positioning of the spray gun 404 at an angle ranging from about 10 degrees to about 80 degrees relative to the plane 900 of the upper portion of the hard shell cluster (i.e. the upper third). Such positioning of the spray gun 404 contributes to an improvement of the sealing and coating abilities of the turbine coating apparatus 100, as it will become apparent below.

Having described the components of the turbine coating apparatus 100 in accordance with one embodiment of the present invention, a method for coating solid forms and more particularly hard shell capsules will now be described in connection with the turbine coating apparatus 100.

According to one embodiment, the turbine coating apparatus 100 is first configured in an idle position for filling the hard shell capsules to be coated in the perforated drum 108. The first lower end 660 of the vertical rod member 408 of the gun support 402 is removed from the central hole 508 of the lower mounting element 500 for removing the gun support 402 from the turbine coating apparatus 100 and facilitating access for feeding the capsules to be coated into the perforated drum 108.

The hard shell capsules are then loaded inside the perforated drum 108 through the opening 334 by the operator. The gun support 402 and the spray gun 404 attached thereto are then repositioned on the turbine coating apparatus 100. More specifically, the first lower end 660 of the vertical rod member 408 is engaged in the central hole 508 of the lower mounting element 500. The first slidable connector 602 and the second slidable connector 622 are then adjusted such that the gun support 402 is positioned eccentrically relative to the rotation axis R¹-R¹. While in this embodiment, the gun support is positioned eccentrically, its position and the position of the spray gun 404 attached thereto is dictated by the size (i.e. diameter) of the turbine coating apparatus and the amount of solid forms contained therein. As such, the gun support 402 may be positioned differently while allowing the spray gun 404 to spray upwardly, as best described below. The nozzle 644 is then positioned relative to the plane 900 of the upper portion of the hard shell capsule bed such that the spray of coating substance is directed upwardly relative to the plane 900 during the operation of the turbine coating apparatus 100 (best shown in FIGS. 9A to 9C). More specifically, the nozzle 644 is positioned for the centerline of the spray (which centerline is designated on FIG. 9A using the reference numeral 902) to define an angle θ¹ relative to the plane 900 of the capsule bed. According to one embodiment, the angle θ¹ preferably ranges between about 10 degrees and about 80 degrees, and more preferably between about 15 degrees and about 50 degrees. Thus, the various components of the spray gun assembly 114 are arranged relative to each other such that the nozzle 644 will define the desired angle θ¹.

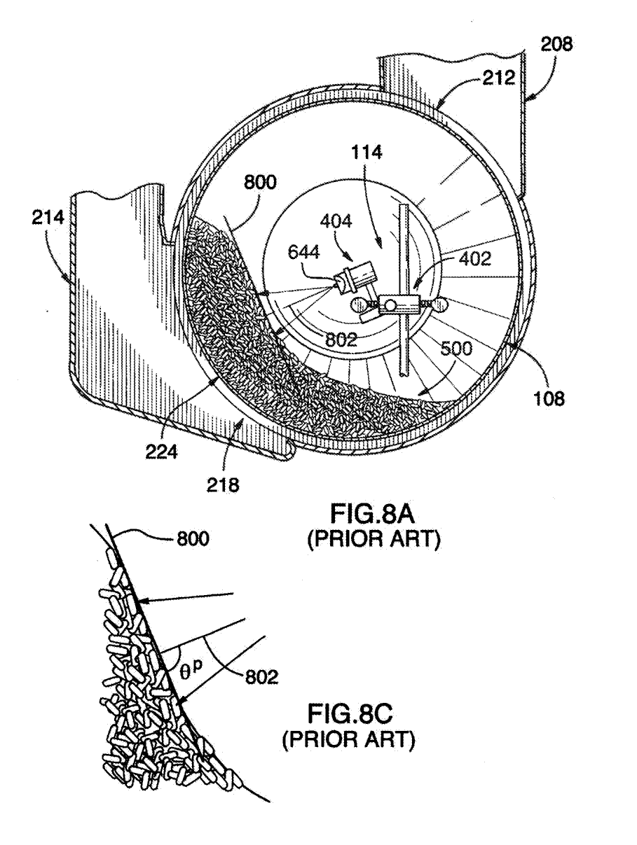

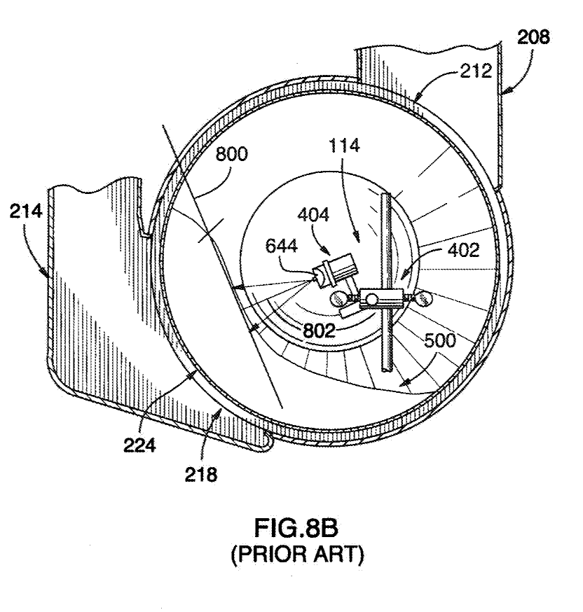

As exemplified below, this position of the spray gun assembly 114 relative to the plane 900 of the hard shell capsule bed provides the turbine coating apparatus 100 with improved coating and sealing capabilities compared to prior art spay assembly configurations. Such a prior art spray gun assembly configuration of the prior art is shown in FIGS. 8A to 8C, where the nozzle 644 of the spray gun assembly 114 is positioned such that the spray of coating substance is directed perpendicularly to the plane 800 of the hard shell capsule bed during the operation of the turbine coating apparatus 100. More specifically, in such prior art configurations, the nozzle 644 is positioned for the centerline of the spray (which centerline is designated on FIG. 8A using the reference numeral 802) to define an angle θ^{P} of about 90 degrees relative to the plane of the capsule bed.

The drive assembly (not shown) is then actuated for urging rotation of the perforated drum 108. In one embodiment, the drive assembly is operable for the perforated drum 108 to rotate in a clockwise direction. A person skilled in the art will appreciate that the perforated drum 108 could be rotated counter-clockwise. In such an embodiment, the capsules gather as a cluster in the bottom right portion of the perforated drum 108, and therefore the position of the nozzle 644 could be mirrored accordingly.

Once rotation of the perforated drum has started, warm air is circulated into the perforated drum. More specifically, warm air is fed into the perforated drum through the second open end 212 of the air intake duct 208 and the perforations 400 of the perforated drum 108 and captured by the second open end 218 of the air exhaust duct 214. A person skilled in the art will appreciate that because the second end 212 of the air intake duct 208 and the second end 218 of the air exhaust duct 214 are radially opposed and the second end 218 of the air exhaust duct 214 is proximal to the bed of capsule during the rotation of the perforated drum 108, the flow of air is caused to percolate or circulate through the cluster of capsules.

Once the capsules have gathered as a cluster in the bottom left portion 224 of the perforated drum 108 due to the rotation thereof, coating material is introduced into the perforated drum 108 through the nozzle 644 of the spray gun 404, as best shown in FIGS. 7 and 9A, until a predetermined weight gain of the hard shell capsules has been achieved.

Once a predetermined amount of weight gain has been provided to the hard shell capsules, the introduction of coating material into the perforated drum 108, the circulation of warm air and the rotation of the perforated drum 108 are stopped for allowing emptying the then coated capsules from the perforated drum 108.

The first lower end 660 of the vertical rod member 408 is then removed from the central 508 of the lower mounting element 500 and the gun support 402 is once again removed from the turbine coating apparatus 100. The coated hard shell capsules are then unloaded from the perforated drum 108 through the opening 334 thereof. Further capsules may now be loaded in the turbine coating apparatus 100 and another cycle of coating may begin.

As it will become apparent from the Examples 1 to 3 that follow, the position of the nozzle 644 relative to the plane of the capsule bed provides the turbine coating apparatus 100 with improved coating and sealing capabilities. In the description and in the following examples, a standard gun position or prior art gun position refers to a nozzle defining about a 90 degrees angle θ^{P} relative to the plane of the capsule bed (e.g. as shown in FIGS. 8A to 8C) while an inverted gun position refers to a nozzle defining an angle θ¹ ranging from about 10 degrees to about 80 degrees relative to the plane of the capsule bed (e.g. as shown in FIGS. 9A to 9C).

### EXAMPLE 1

### Material and Methods

A first comparative study was performed using 4.0 kg of vegetal hard shell capsules Vcaps™ #0 manufactured by Capsugel (Greenwood, S.C., USA), The vegetal hard shell capsules were divided into two lots, Lots 1A and 1B, each lot comprising 2.0 kg of hard shell capsules. The vegetal hard shell capsules Vcaps™ #0 were coated with a sub-coating layer of Spectrablend #50846 manufactured by Sensient Technologies Canada (Mississauga, ON, Canada), followed by a coating using an enteric coating of Eudragit L 30 D-55 manufactured by Röhm GmbH (Darmstadt, Germany). The parameters of this experiment are summarized below in the following TABLE 1:

| | Lot 1A | Lot 1B |
|---|---|---|
| Sub-coating | | |
| Coating ingredient | Spectrablend | Spectrablend |
| | #50846 ™ | #50846 ™ |
| Ingredient | 10% | 10% |
| Concentration (w/v) | | |
| Spray gun | Schlick # 970/7-1 S75 | Schlick # 970/7-1 S75 |
| | with Anti-Bearding | with Anti-Bearding |
| | Cap, nozzle diameter | Cap, nozzle diameter |
| | of 1.0 mm, flow rate | of 1.0 mm, flow rate |
| | from 10 g/min to | from 10 g/min to |
| | 30 g/min | 30 g/min |
| Number of guns | 2 | 2 |
| Gun position | Standard | Inverted |
| Atomization pressure | 20 | 20 |
| (psi) | | |
| Pressure pattern (psi) | 25 | 25 |
| Air flow (cfm) | 180 | 180 |
| Pump flow (g/min)³ | 12.9 | 12.7 |
| Coating time | 92:22 | 93:48 |
| (min:sec) | | |
| Weight increase (%) | 6 | 6 |
| Enteric Coating | | |
| Coating ingredient | Eudragit ® | Eudragit ® |
| | L 30 D-55 | L 30 D-55 |
| Ingredient | 20% | 20% |
| Concentration (w/v) | | |
| Spray gun | Spraying System Co. | Spraying System Co. |
| | #1/4 JAU-SS with cap | #1/4 JAU-SS with cap |
| | #134255-45-SS, | #134255-45-SS, |
| | nozzle #60100-SS | nozzle #60100-SS |
| | diameter of 1.3 mm | diameter of 1.3 mm |
| Number of guns | 1 | 1 |
| Gun position | Standard | Inverted |
| Atomization pressure | 30 | 25 |
| (psi) | | |
| Pressure pattern (psi) | 30 | 25 |
| Air flow (cfm) | 180 | 180 |
| Pump flow (g/min)³ | 14.0 | 15.9 |
| Coating time | 71:20 | 62:45 |
| (min:sec) | | |
| Weight increase | 10 | 10 |

Samples of twenty hard shell capsules were collected from the turbine coating apparatus 100 from each Lot 1A and 1B at five different stages of the enteric coating process, for a total of ten samples. More specifically, samples were collected at five different stages of the coating process bases on the theoretical weight gain, as set out in TABLE 2 below:

| Theoretical Weight gain | Lot 1A | Lot 1B |
|---|---|---|
| Sub-coating 6% | Sample 1000a | Sample 1000b |
| Sub-coating 6% + | Sample 1002a | Sample 1002b |
| enteric coating 6% | | |
| Sub-coating 6% + | Sample 1004a | Sample 1004b |
| enteric coating 8% | | |
| Sub-coating 6% + | Sample 1006a | Sample 1006b |
| enteric coating 9% | | |
| Sub-coating 6% + | Sample 1008a | Sample 1008b |
| enteric coating 10% | | |

The weight of every hard shell capsule from each sample was measured to assess the actual weight gain and the results were averaged for ensuring that variations of the quality of sealing of the capsules noted between Lot 1A and Lot 1B could not be attributable to variation of the actual or empirical weight gain at the various stages of the coating process. The same samples were then visually examined using a stereoscope to assess the quality of the sealing of each capsule and the variability of such sealing quality.

### RESULTS

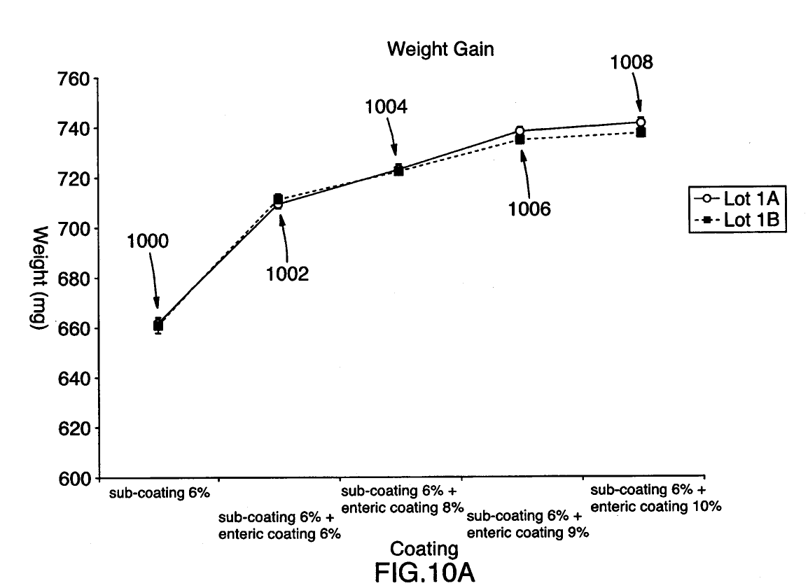

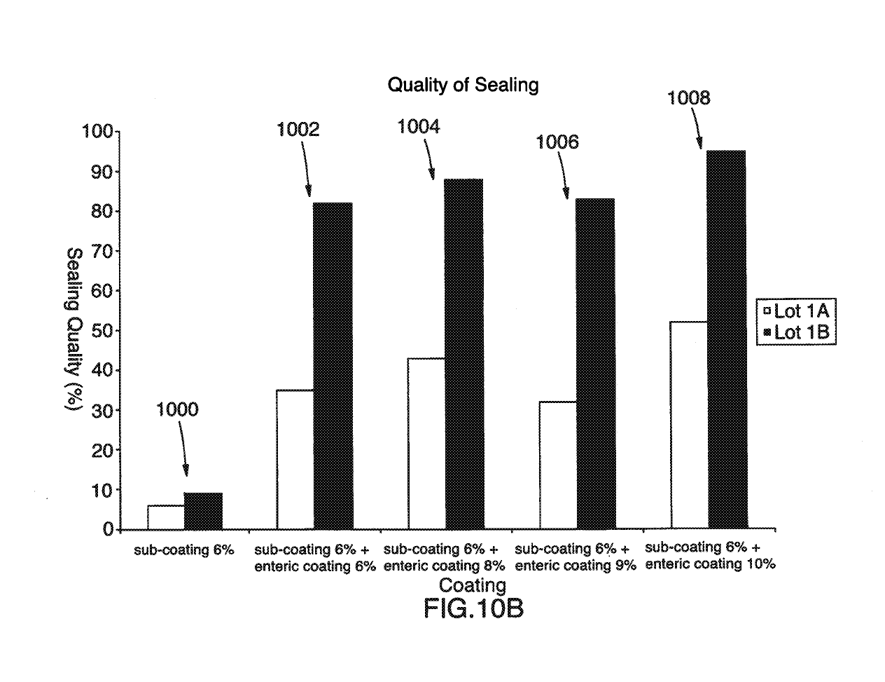

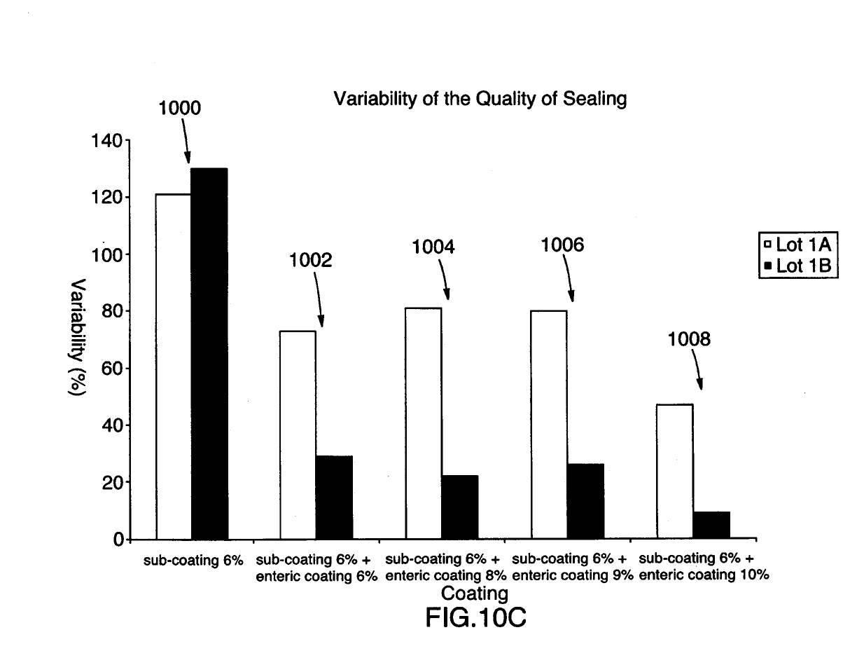

The results of the experiment conducted for are shown in FIGS. 10A to 10C. FIG. 10A shows that the empirical or actual weight gain of the capsules sampled at the various stages of the coating process does not vary significantly between Lot 1A and Lot 1B.

Turning now to FIG. 10B, capsules from sample 1000a and 1000b, (i.e. only comprising sub-coating), show a relatively low quality of sealing. This low quality of sealing of sample 1000, namely about 5% for Lot 1A and about 10% for Lot 1B, reflects that even at a very early stage of the coating process, the configuration of the spray gun assembly of the present invention (i.e. the inverted position) exhibit better sealing capabilities than the configuration of the spray gun assembly taught by the prior art (i.e. the standard position).

The better sealing capabilities of the spray gun assembly configured in the inverted position is further shown at all other various stages of the coating process, where the inverted gun position provides about twice the quality of sealing compared to an enteric coating applied with a standard gun position. For instance, upon completion of the coating process, capsules from sample 1008b collected from Lot 1B showed an averaged quality of sealing of 95% while capsules from sample 1008a collected from Lot 1A present an averaged quality of sealing of 52%, which is typical with such a prior art spray gun configuration (i.e. the standard spray gun configuration). Since the same amount of coating material is used for each sample of both Lots 1A and 1B, the inversion of the spray gun thus greatly improves the quality of sealing of capsules without increasing production costs.

Furthermore, capsules coated with an enteric coating providing a first theoretical weight increase, of 6% for instance, using a spray gun in a inverted gun position (i.e. sample 1002b) present a better quality of sealing than capsules coated with an enteric coating providing a second, higher theoretical weight increase, of 10% using a spray gun in a standard gun position (i.e. sample 1008a). This tend to suggest that a turbine coating apparatus configured in the inverted position according to the present invention would provide improved quality of sealing for hard shell capsules while using less coating material, thereby lowering production costs.

Results obtained with samples 1000a to 1008b not only shown that the overall quality of sealing of the capsules is improved by providing a spray gun assembly in inverted position. Indeed, results of the experiments conducted show that the variability of the quality of sealing is also better where the spray gun assembly is in inverted position as compared to the standard position (shown in FIG. 10C). Such a decrease in the variability of the sealing quality implies less hard shell capsules being discarded due to poor quality of sealing or needing to be recoated, thus improving efficiency of the coating process.

In summary, results of the experiments conducted shown that with comparable weight gain, the quality of sealing and variability thereof are improved where the spray gun assembly is configured according to the present invention as compared to a spray gun assembly configured according to the teachings of the prior art (i.e. the standard position). Thus, inversion of the spray gun assembly (i.e. positioning the spray gun for defining an angle ranging between about 10 degrees and about 80 degrees) tends to increase the overall efficiency of the coating process.

### EXAMPLE 2

### Material and Methods

A second comparative study was performed using 4.0 kg of vegetal hard shell capsules Vcaps™#0 manufactured by Capsugel (Greenwood, S.C., USA). The vegetal hard shell capsules were divided into two lots, Lot 2A and Lot 2B, each lot comprising 2.0 kg of hard shell capsules. While the experiments conducted were similar to of Example 1, the spray gun and the sub-coating ingredient were modified. The parameter of the experiments conducted for Example 2 are summarized in TABLE 3 below:

| | Lot 2A | Lot 2B |
|---|---|---|
| Sub-coating | | |
| Coating ingredient | Spectrablend | Spectrablend |
| | #50844 ™ | #50844 ™ |
| Ingredient | 14% | 14% |
| Concentration (w/v) | | |
| Spray gun | Schlick #930/7-1 S35 | Schlick #930/7-1 S35 |
| | with Anti-bearding | with Anti-Bearding |
| | Cap, nozzle diameter | cap, nozzle diameter |
| | of 1.2 mm, flow rate | of 1.0 mm, flow rate |
| | from 30 g/min to | from 10 g/min to |
| | 120 g/min | 30 g/min |
| Number of guns | 1 | 1 |
| Gun position | Standard | Inverted |
| Atomization pressure | 15 | 15 |
| (psi) | | |
| Pressure pattern (psi) | 20 | 20 |
| Air flow (cfm) | 180 | 180 |
| Pump flow (g/min)³ | 13.5 | 13.8 |
| Coating time | 31:58 | 31:20 |
| (min:sec) | | |
| Weight increase (%) | 3 | 3 |
| Enteric Coating | | |
| Coating ingredient | Eudragit ® | Eudragit ® |
| | L 30 D-55 | L 30 D-55 |
| Ingredient | 20% | 20% |
| Concentration (w/v) | | |
| Spray gun | Schlick #930/7-1 S35 | Schlick #930/7-1 S35 |
| | with Anti-Bearding | with Anti-Bearding |
| | Cap, nozzle diameter | Cap, nozzle diameter |
| | of 1.2 mm, flow rate | of 1.2 mm, flow rate |
| | from 30 g/min to | from 30 g/min to |
| | 120 g/min | 120 g/min |
| Number of guns | 1 | 1 |
| Gun position | Standard | Inverted |
| Atomization pressure | 15 | 15 |
| (psi) | | |
| Pressure pattern (psi) | 20 | 20 |
| Air flow (cfm) | 180 | 180 |
| Pump flow (g/min)³ | 13.8 | 14.6 |
| Coating time | 72:12 | 71:04 |
| (min:see) | | |
| Weight increase (%) | 10 | 10 |

Similarly to Example 1, samples of twenty hard shell capsules were collected from the turbine coating apparatus from both lots 2A and 2B at different stages of the enteric coating process. In total, fourteen samples were collected in Example 2, seven in each of the two lots 2A and 2B, as summarized in TABLE 4 below:

| Theoretical Weight gain | Lot 2A | Lot 2B |
|---|---|---|
| Sub-coating 3% | Sample 1100a | Sample 1100b |
| Sub-coating 3% + | Sample 1102a | Sample 1102b |
| enteric coating 5% | | |
| Sub-coating 3% + | Sample 1104a | Sample 1104b |
| enteric coating 6% | | |
| Sub-coating 3% + | Sample 1106a | Sample 1106b |
| enteric coating 7% | | |
| Sub-coating 3% + | Sample 1108a | Sample 1108b |
| enteric coating 8% | | |
| Sub-coating 3% + | Sample 1110a | Sample 1110b |
| enteric coating 9% | | |
| Sub-coating 3% + | Sample 1112a | Sample 1112b |
| euteric coating 10% | | |

The weight of every capsule from each sample was measured to assess the actual weight gain and the results were averaged for ensuring that variations of the quality of sealing of the capsules noted between Lot 2A and Lot 2B could not be attributable to variation of the actual or empirical weight gain at the various stages of the coating process. The same samples were then visually examined using a stereoscope to assess the quality of the sealing of each capsule and the variability of such sealing quality.

### RESULTS

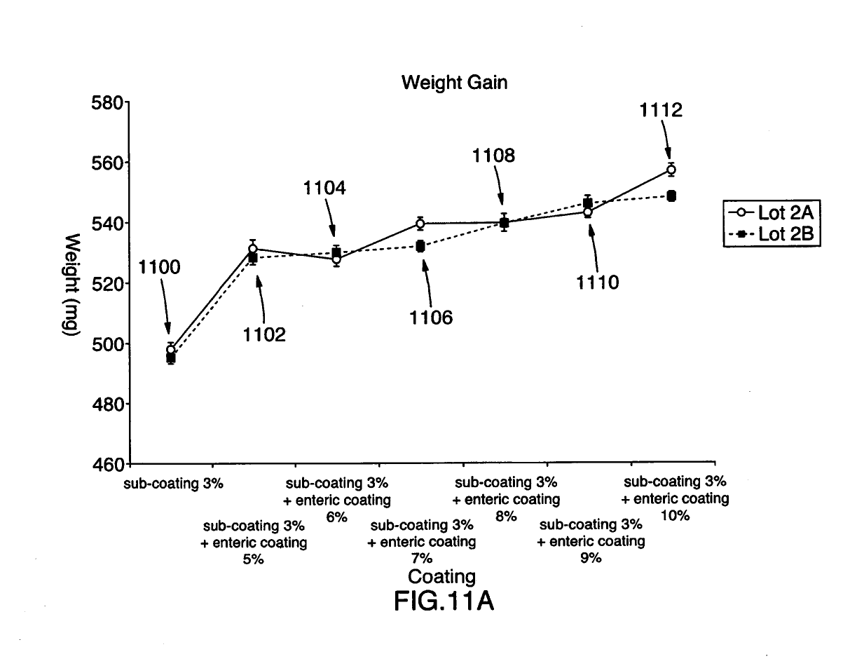

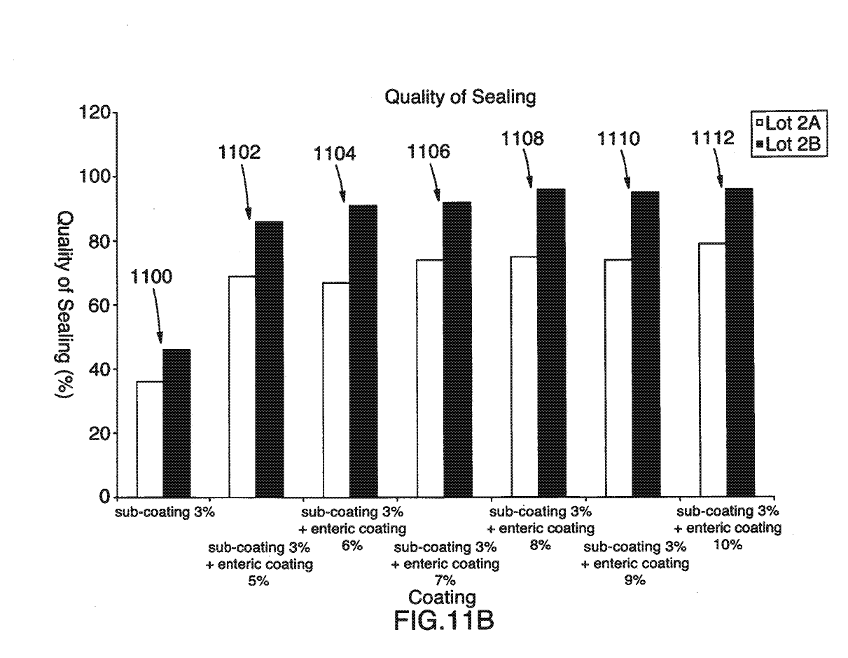

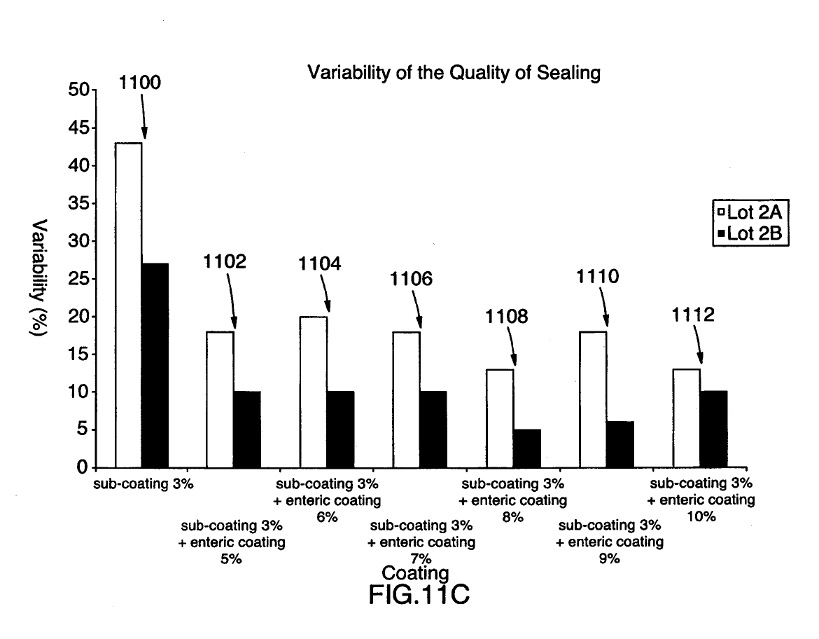

The results of the experiment conducted for are shown in FIGS. 11A to 11C. Similarly to Example 1, FIG. 11A shows that the empirical or actual weight gain of the capsules sampled at the various stages of the coating process does not vary significantly between Lot 2A and Lot 2B.

While the difference of the quality of sealing observed between Lot 2A and Lot 2B throughout the experiments conducted for the purpose of Example 2 was less important than the difference of the quality of sealing observed between Lot 1A and Lot 1B of Example 1, FIG. 11B stills shows that the quality of the sealing was significantly improved. Furthermore, the variability of the quality of sealing was significantly improved using the spray gun in inverted configuration as compared to the spray gun in the standard configuration, as best shown in FIG. 11C. Results from this experiment show that the type of spray gun used for applying the enteric coating or the characteristics of the sub-coating layer do not affect the improvement provided by providing a spay gun configured in accordance with the present invention.

### EXAMPLE 3

### Material and Methods

A third comparative study was performed to assess whether the use of gelatine capsules instead of vegetal capsules may impair the overall benefits if using a spray gun assembly in inverted position rather than in standard position. Thus, for the purpose of Example 3, 4.0 kg of gelatin hard shell capsules Coni-Snap™ #0 manufactured by Capsugel (Greenwood, S.C., USA) were used. The gelatine hard shell capsules were divided into two lots, Lot 3A and Lot 3B, each lot comprising 2.0 kg of hard shell capsules. The spray gun 404 and the sub-coating ingredient were identical to those used in Example 2, as be described in TABLE 5 below:

| | Lot 3A | Lot 3B |
|---|---|---|
| Sub-coating | | |
| Coating ingredient | Spectrablend | Spectrablend |
| | #50844 ™ | #50844 ™ |
| Ingredient | 14% | 14% |
| Concentration (w/v) | | |
| Spray gun | Schlick #930/7-1 S35 | Schlick #930/7-1 S35 |
| | with Anti-bearding | with Anti-Bearding |
| | Cap, nozzle diameter | cap, nozzle diameter |
| | of 1.2 mm, flow rate | of 1.0 mm, flow rate |
| | from 30 g/min to | from 10 g/min to |
| | 120 g/min | 30 g/min |
| Number of guns | 1 | 1 |
| Gun position | Standard | Inverted |
| Atomization pressure | 15 | 15 |
| (psi) | | |
| Pressure pattern (psi) | 20 | 20 |
| Air flow (cfm) | 180 | 180 |
| Pump flow (g/min)³ | 13.6 | 13.6 |
| Coating time | 31:29 | 31:32 |
| (min:sec) | | |
| Weight increase | 3 | 3 |
| Enteric Coating | | |
| Coating ingredient | Eudragit ® | Eudragit ® |
| | L 30 D-55 | L 30 D-55 |
| Ingredient | 20% | 20% |
| Concentration (w/v) | | |
| Spray gun | Schlick #930/7-1 S35 | Schlick #930/7-1 S35 |
| | with Anti-bearding | with Anti-Bearding |
| | Cap, nozzle diameter | cap, nozzle diameter |
| | of 1.2 mm, flow rate | of 1.0 mm, flow rate |
| | from 30 g/min to | from 10 g/min to |
| | 120 g/min | 30 g/min |
| Number of guns | 1 | 1 |
| Gun position | Standard | Inverted |
| Atomization pressure | 15 | 15 |
| (psi) | | |
| Pressure pattern (psi) | 20 | 20 |
| Air flow (cfm) | 180 | 180 |
| Pump flow (g/min)³ | 14.0 | 14.2 |
| Coating time | 71:08 | 70:30 |
| (min:sec) | | |
| Weight increase (%) | 10 | 10 |

Again, samples of twenty capsules were collected from the turbine coating apparatus from both lots 3A and 3B at different stages of the enteric coating process. In total, fourteen samples were collected in Example 3, seven in each of the two lots 3A and 3B, as summarized in TABLE 6 below:

| Theoretical Weight gain | Lot 3A | Lot 3B |
|---|---|---|
| Sub-coating 3% | Sample 1200a | Sample 1200b |
| Sub-coating 3% + | Sample 1202a | Sample 1202b |
| enteric coating 5% | | |
| Sub-coating 3% + | Sample 1204a | Sample 1204b |
| enteric coating 6% | | |
| Sub-coating 3% + | Sample 1206a | Sample 1206b |
| enteric coating 7% | | |
| Sub-coating 3% + | Sample 1208a | Sample 1208b |
| enteric coating 8% | | |
| Sub-coating 3% + | Sample 1210a | Sample 1210b |
| enteric coating 9% | | |
| Sub-coating 3% + | Sample 1212a | Sample 1212b |
| enteric coating 10% | | |

The weight of every capsule from each sample was measured to assess the actual weight gain and the results were averaged for ensuring that variations of the quality of sealing of the capsules noted between Lot 3A and Lot 3B could not be attributable to variation of the actual or empirical weight gain at the various stages of the coating process. The same samples were then visually examined using a stereoscope to assess the quality of the sealing of each capsule and the variability of such sealing quality.

### RESULTS

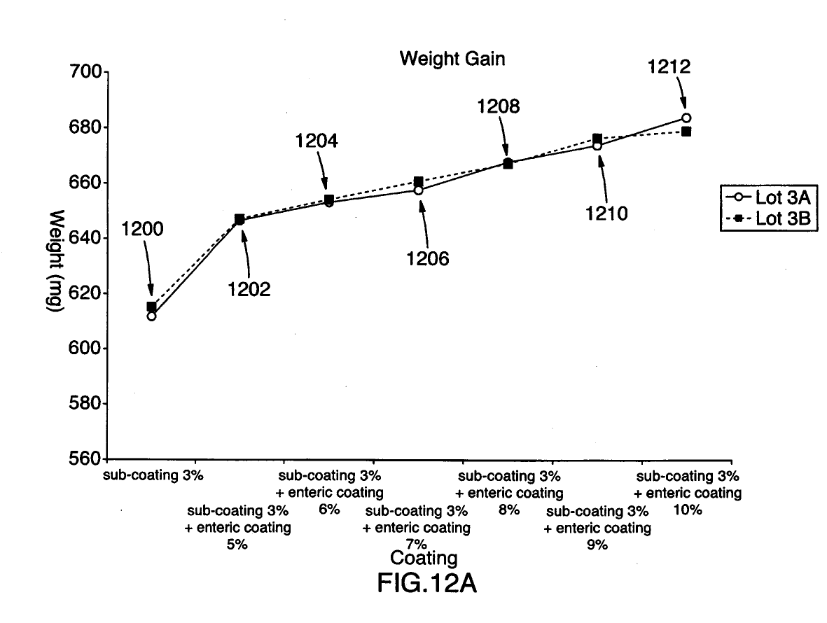

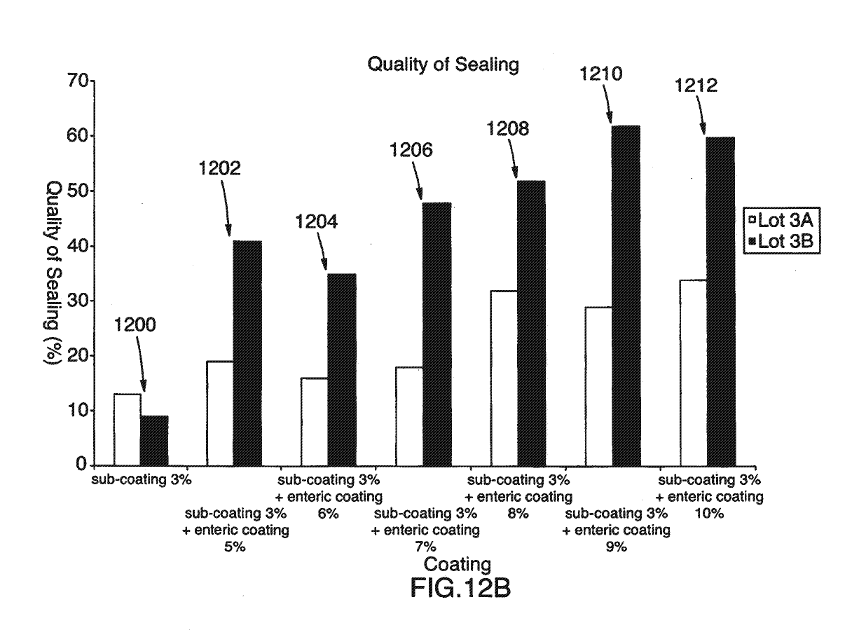

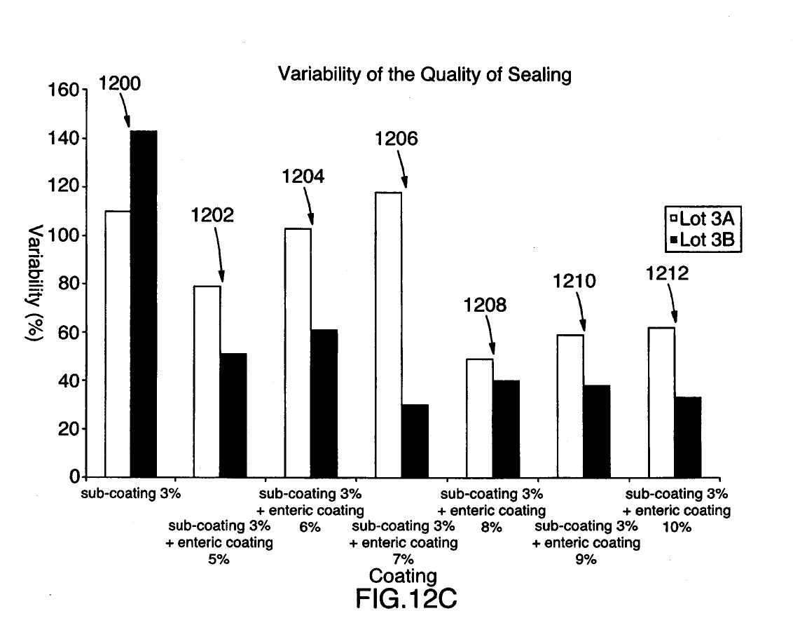

The results of the experiment conducted for are shown in FIGS. 12A to 12C. Similarly to Example 1 and Example 2, FIG. 12A shows that the empirical or actual weight gain of the capsules sampled at the various stages of the coating process does not vary significantly between Lot 3A and Lot 3B.

With reference to FIGS. 12B and 12C, the quality of sealing and the variability of sealing quality are greatly improved using the spray gun in inverted position as compared to a spray gun in standard position. This shows that the spray gun configuration of the present invention enhance the overall coating process with both vegetal and gelatin capsules.

Although the foregoing description and accompanying drawings relate to specific preferred embodiments of the present invention as presently contemplated by the inventor, it will be understood that various changes, modifications and adaptations, may be made.

1-12. (canceled)

13. A turbine coating apparatus provided with a perforated drum rotatably mounted in a housing, the rotation of said perforated drum gathering solid forms in a cluster defining a coating plane, said turbine coating apparatus comprising:
a gun support mounted to said turbine coating apparatus; and
at least one spray gun mounted to said gun support for providing a spray of a coating substance onto said cluster of solid forms, said at least one spray gun being positioned into said perforated drum in rotation for said spray of a coating substance to define a spray angle of less than 90 degrees relative to said coating plane defined by said cluster of solid forms.

14. The turbine coating apparatus as claimed in claim 13, wherein said spray angle ranges from about 10 degrees to about 80 degrees.

15. The turbine coating apparatus as claimed in claim 14, wherein said spray angle comprises a spray angle ranging from about 15 degrees to about 50 degrees.

16. The turbine coating apparatus as claimed in claim 13, wherein said gun support comprises a first end mountable to said housing of said apparatus and a second end, said at least one spray gun being mounted between said first end and said second end of said gun support.

17. The turbine coating apparatus as claimed in claim 16, wherein said at least one spray gun is adjustably positionable between said first end and said second end of said gun support.

18. The turbine coating apparatus as claimed in claim 13, wherein said solid forms are selected from the group consisting of capsules, granules and tablets.

19. The turbine coating apparatus as claimed in claim 18, wherein said capsules comprise hard shell capsules.

20. The turbine coating apparatus as claimed in claim 19, wherein said hard shell capsules comprise vegetal capsules.

21. The turbine coating apparatus as claimed in claim 19, wherein said hard shell capsules comprise gelatine capsules.

22. The turbine coating apparatus as claimed in claim 18, wherein said capsules comprise capsules housing a substance selected from the group consisting of a probiotic culture, a pharmaceutical compound, a nutraceutical, a dietary supplement, a vitamin and a veterinary compound.

23. The turbine coating apparatus as claimed in claim 13, wherein said coating substance is selected from the group consisting of a sub-coating substance, an enteric coating substance and a film coating substance.

24. The turbine coating apparatus as claimed in claim 13, wherein said at least one spray gun is selected from the group consisting of a Schlick #930/7-1 S35™ spray gun, a Schlick #970/7-1 S75™ spray gun and a Spraying System Co. #1/4 JAU-SS™ spray gun.

25. A method for coating and sealing solid forms, said method comprising:
(a) providing a turbine coating apparatus comprising a perforated drum rotatably mounted in a housing;
(b) providing at least one spray gun positionable into said perforated drum for providing a spray of a coating substance;
(c) loading said solid forms into said perforated drum;
(d) urging rotation of said perforated drum, said rotation of said perforated drum gathering solid forms in a cluster defining a coating plane;
(e) positioning said at least one spray gun into said perforated drum for said spray of a coating substance to define a spray angle of less than 90 degrees relative to said coating plane;
(f) providing said spray of a coating substance onto said cluster of solid forms until a predetermined amount of weight gain has been provided to said solid forms; and
(g) collecting the coated solid forms.

26. The method as claimed in claim 25, wherein said spray angle ranges from about 10 degrees to about 80 degrees.

27. The method as claimed in claim 26, wherein said spray angle ranges from about 15 degrees to about 50 degrees.

28. The method as claimed in claim 25 further comprising providing a gun support mounted to said turbine coating apparatus, said at least one spray gun being mounted to said gun support.

29. The method as claimed in claim 28, wherein said gun support comprises a first end mountable to said housing of said apparatus and a second end, said at least one spray gun being mounted between said first end and said second end of said gun support.

30. The method as claimed in claim 29, wherein positioning said at least one spray gun comprises adjustably positioning said at least one spray gun between said first end and said second end of said gun support.

31. The method as claimed in claim 25, wherein positioning said at least one spray gun further comprises positioning said at least one spray gun eccentrically relative to a rotation axis of said perforated drum.

32. The method as claimed in claim 25, wherein said solid forms are selected from the group consisting of capsules, granules and tablets.

33. The method as claimed in claim 32, wherein said capsules comprise hard shell capsules.

34. The method as claimed in claim 33, wherein said hard shell capsules comprise vegetal capsules.

35. The method as claimed in claim 33, wherein said hard shell capsules comprise gelatine capsules.

36. The method as claimed in claim 32, wherein said capsules comprise capsules housing a substance selected from the group consisting of a probiotic culture, a pharmaceutical compound, a nutraceutical, a dietary supplement, a vitamin and a veterinary compound.

37. The method as claimed in claim 25, wherein said coating substance is selected from the group consisting of a sub-coating substance, an enteric coating substance and a film coating substance.

38. The method as claimed in claim 25, wherein said at least one spray gun is selected from the group consisting of a Schlick #930/7-1 S35™ spray gun, a Schlick #970/7-1 S75™ spray gun and a Spraying System Co. #1/4 JAU-SS™ spray gun.

39. The method as claimed in claim 26, wherein loading said solid forms comprises removing said gun support from said turbine coating apparatus for facilitating access to said perforated drum and mounting said gun support to said turbine coating apparatus once said solid forms have been loaded into said perforated drum.

40. The method as claimed in claim 26, wherein said coated solid forms collecting comprises removing said gun support from said turbine coating apparatus for facilitating access to said perforated drum and removing said coated solid forms from said perforated drum.

41. A spray gun assembly for a turbine coating apparatus, according to claim 13.
